(19) United States
(12) Patent Application Publication
(10) Pub. No.: US 2023/0381352 A1
(43) Pub. Date: Nov. 30, 2023

(54) SYSTEM, DEVICE, AND METHOD FOR MITIGATING BACTERIAL BIOFILMS ASSOCIATED WITH INDWELLING MEDICAL DEVICES

(71) Applicant: NANOVIBRONIX, INC., Elmsford, NY (US)

(72) Inventor: Brian Murphy, Fort Lauderdale, FL (US)

(21) Appl. No.: 17/646,715

(22) Filed: Dec. 31, 2021

(51) Int. Cl. A61L 2/025; A61L 2/24

(52) U.S. Cl. CPC A61L 2/025; A61L 2/24; A61L 2202/24

(57) ABSTRACT

A system, device and methods for inhibiting biofilm formation and preventing infection associated with indwelling medical devices by generating and applying surface acoustic waves (SAW) to a surface of the indwelling medical device. One or more piezoelectric ultrasound transducer(s) in direct contact with a surface of the implantable medical device generates high frequency mechanical vibrations in a range from KHz to MHz upon receipt of an electrical signal from a processor. The high frequency mechanical vibrations produce SAW in the nanometer range along a surface of the indwelling medical device and inhibit formation of bacterial biofilms.

### FIELD

Embodiments of the present disclosure relate generally to preventing infections associated with indwelling medical devices by inhibition of bacterial biofilms. More specifically, indwelling medical devices, systems and methods of the present disclosure mitigate and/or prevent biofilm formation on a surface of the subject indwelling medical device through the application of surface acoustic waves thereto.

### BACKGROUND

Infections associated with indwelling devices that are inserted into more than 25% of patients during hospitalization constitute a major cause of morbidity and mortality in hospitalized patients and significantly increase medical costs. Based on the CDC's annual healthcare-associated infection (HAI) prevalence survey, surgical site infections, which include infections stemming from indwelling devices, account for 20% of all HAIs, are associated with a 2 to 11-fold increase in the risk of mortality, extend patients' hospital stays by 10 days on average, and increase hospitalization costs by more than $20,000 per admission. Examples of common indwelling medical devices include, without limitation, urinary, endotracheal, intravenous, and implants. The incidence of bacterial infections resulting from biofilm formation in such patients is approximately 5-10% per day, with virtually all patients undergoing long-term treatment (28 days or more) with said indwelling device.

The first stage in biofilm formation from planktonic microorganisms is adhesion to solid surfaces. Adhesion stimulates bacterial aggregation and proliferation forming micro-colonies. The colonies excrete an encasing polysaccharide ‘slime’ which consolidates their attachment to surfaces and the microaggregates differentiate into characteristic biofilms. The encasing extracellular polysaccharide matrix regulates the exchange of ions and nutrients between biofilms and their surrounding environment. Microbial biofilms form when bacteria adhere to a hydrated surface, encasing themselves in a protective extracellular matrix (ECM), which increases their resistance to antimicrobial treatments. In comparison to their planktonic counterparts, bacteria in biofilms require 500-5000 times higher doses of antibiotics for removal. Moreover, because bacterial cells may slough off from mature biofilms and spread throughout the environment, biofilms can also serve as a source of recurring healthcare-associated infections, particularly when formed on indwelling medical devices. Urinary catheters, for example, exhibit an infection rate of 5-7% per day after implantation due to colonization by bacterial biofilms and leading to catheter-associated urinary tract infections (CAUTIs). Microbial biofilms also present serious challenges to the immune system because the expression of bacterial antigens within the ECM is suppressed, and the colony structures are highly resistant to phagocytosis. Altogether, these properties render biofilms exceedingly difficult to eradicate and explain the severity, persistence, and high levels of morbidity associated with infections produced by biofilms.

The severe and potentially fatal consequences of microbial biofilm infections have generated efforts to prevent biofilm formation, particularly on indwelling devices. Catheters coated hydrogel, silver salts, and anti-microbials have been proposed, but provide minimal reduction in infection incidence rates.

Studies have shown that the addition of low-frequency ultrasound simultaneously with the application of antibiotics enhances the effectiveness of the antibiotics in killing the bacteria. Whereas ultrasound by itself was not found to have any significant effect on the bacteria, applying ultrasound in conjunction with antibiotics resulted in a significantly greater fraction of bacteria being killed than when using the antibiotic alone. than when the antibiotics were applied alone.

Mechanical approaches to preventing biofilm formation have utilized ultrasonic energy, but the focus to date has been predominantly on increasing biofilm sensitivity to antibiotics. Moreover, although the combination of ultrasound with antibiotics has been found effective in the reduction of E. Coli biofilm in animal models, it falls short of providing a comprehensive solution to the biofilm problem. In the methods, the aim has been to clean the medical device from contaminants and accumulated deposits, rather than fighting against the initial access of bacteria which is the adhesion of bacteria to the device surface.

While advances have been made in infection control practices, including improved operating room ventilation, sterilization methods, barriers, surgical technique, and availability of antimicrobial prophylaxis, infections from biofilms associated with indwelling medical devices remain a substantial cause of morbidity, prolonged hospitalization, and even death. Benefits of the present invention include a reduction or even prevention of infections commonly resulting from biofilm formation on surfaces of indwelling devices.

### SUMMARY

Ultrasound has been shown to interfere with bacterial colonization and to reduce formation of bacterial biofilm. Specifically, it has been shown that ultrasound interferes with bacterial touch sensors that facilitate bacterial docking for formation of biofilms. In addition to improved indwelling medical devices, the present invention provides for an ultrasound system that effectively transforms the indwelling medical device into a therapeutic device by generation of surface acoustic waves (SAW) along a surface of the indwelling medical device to inhibit biofilm formation and prevent biofilm-associated infection.

In embodiments, a portable ultrasound system comprises: an energy generating module operative to generate a driving signal that can be transformed into ultrasonic energy, wherein said energy generating module comprises a power source, an oscillator, and a driver component; and an ultrasound transducer comprising a piezoelectric component, said ultrasound transducer being operative to receive the driving signal from the energy generating module, to transform the driving signal into ultrasonic energy, and to control a direction of the ultrasonic energy emitted from the ultrasound transducer, wherein the oscillator and driver component are housed on or within the ultrasound transducer, and the power source is not housed on or within the ultrasound transducer.

In embodiments, ultrasound energy from the ultrasound transducer may be emitted as pulsed, continuous, or both pulsed and continuous ultrasonic energy.

In embodiments, the energy generating module comprises a voltage controller operative to control power distribution from the power source to the oscillator and driver component. The voltage controller comprises an on/off controller switch coupled to a transistor switch. The energy generating module and the ultrasound transducer of the present invention may be operatively connected and the driving signal may be wirelessly communicated to the ultrasound transducer without a bus.

Also provided is an assembly of an indwelling medical device and the portable ultrasound system of the invention, wherein the indwelling medical device is selected from a PEG tube an IV catheter and comprises the portable ultrasound transducer coupled to an exterior surface thereof. In embodiments, the ultrasound transducer comprises a plurality of ultrasound transducers configured to supply SAW in a plurality of directions.

In further embodiments, provided is a portable ultrasound device comprising: a power source; an ultrasound driver; and an ultrasound transducer configured to receive and convert a driving signal from the ultrasound driver into low frequency surface acoustic waves, wherein the portable ultrasound device is comprised by an indwelling medical device and the surface acoustic waves generated by the ultrasound transducer are transmitted along a surface of the indwelling medical device. The portable ultrasound device may be coupled to an exterior surface of the indwelling medical device and transmits the surface acoustic waves along an exterior surface of the indwelling medical device in an elongated direction thereof. In certain embodiments, the indwelling medical device is an intravenous (IV) catheter or a PEG tube.

The ultrasound transducer according to embodiments of the invention may comprise a plurality of ultrasound transducers. In preferred embodiments, the portable ultrasound device of the invention comprises a thin plate piezo transducer in direct contact with a surface of the indwelling medical device, wherein the thin plate piezo transducer is configured to vibrate in bending vibration modes to create standing acoustic waves upon activation by a processor.

In still further embodiments, the portable ultrasound device may be configured within a patch for adhesion of an indwelling medical device to a body of a patient. In other embodiments, the ultrasound is implantable within a subject as part of an indwelling medical device.

In a further aspect of the invention, provided is an improved indwelling medical device for implantation in a subject's body, the indwelling medical device having an elongated tube shape comprising a proximal end opposite a direction of the body and a distal end in the direction of the body, and comprising the portable ultrasound system operatively coupled thereto, wherein the portable ultrasound transducer is configured within a surface of the indwelling medical device. In embodiments, an indwelling medical device comprises the portable ultrasound device operatively coupled thereto, wherein the ultrasound transducer comprises a piezoelectric element in direct contact with the surface of the indwelling medical device and, upon receipt of the electrical signal, generates high frequency mechanical vibrations to create surface acoustic waves in the nanoscale range on an internal surface and external surface of the indwelling medical device.

Also included and described herein are methods for inhibiting formation of bacterial biofilm associated with indwelling medical devices. In embodiments, a method for inhibiting formation of bacterial biofilms comprises: providing an indwelling medical device, an ultrasound transducer positioned on the indwelling medical device, wherein the ultrasound transducer comprises a piezoelectric material in direct contact with a surface of the indwelling medical device, and a processor in electrical communication with the ultrasound transducer; positioning at least a distal end of the indwelling medical device in a body of a subject; activating the ultrasound transducer by powering the processor, wherein activating the ultrasound transducer produces elliptical motion of particles and generates surface acoustic waves along a surface of the indwelling medical device; and controlling parameters of the ultrasound transducer via the processor such that a vibration amplitude of the bacteria is smaller than a X-potential repulsive zone of the bacteria.

### BRIEF DESCRIPTION OF THE DRAWINGS

The present invention may be better understood by referring to the following description in conjunction with the accompanying drawings in which:

FIG. 1 is a schematic illustration of an indwelling medical device with an ultrasound transducer for producing surface acoustic waves (SAW) according to embodiments of the invention;

FIG. 2 is a block diagram illustration of a system for generating SAW on a surface of an indwelling medical device to inhibit biofilm formation in accordance with embodiments of the invention;

FIG. 3 is an illustration of a portable ultrasound system according to a wireless embodiment of the invention;

FIG. 4A is an illustration of an ultrasound transducer according to an embodiment of the invention, wherein the base portion of the ultrasound transducer comprises a piezo-element and acts as the activating portion;

FIG. 4B is an illustration of the ultrasound transducer of FIG. 4A during vibrations upon activation;

FIG. 5 is a schematic illustration depicting elliptical motion of SAW generated on a surface of an indwelling medical device according to embodiments of the invention; and

FIG. 6 is an illustration of an ultrasound system according to embodiments of the invention coupled to an indwelling medical device that is inserted into a body of a patient.

FIG. 7 is an illustration of the ultrasound transducer in a patch configuration according to an embodiment of the invention.

### DETAILED DESCRIPTION

The embodiments described herein are illustrative of the invention and not intended to limit the scope of the invention as encompassed by the appended claims. Various modifications to the described embodiments will be apparent to those skilled in the art and the general principles defined herein may be applied to other embodiments.

The term “biofilm” as used herein may encompass microbes, microorganisms, viruses, fungi, deposits, particles, pathogenic organisms, cells, and other bioactive materials.

The term “surface acoustic waves” or “SAW” as used herein include several types of waves or combinations thereof as follows: surface waves, including Rayleigh (elliptical orbit, symmetrical mode); plate waves, including Lamb (component perpendicular to surface) and Love (parallel to plane layer, perpendicular to wave direction), and Stoneley (Leaky Rayleigh Waves); waves guided along interface; and Sezawa— antisymmetric mode waves. Surface or Rayleigh waves travel along the boundary between two different media, penetrating to a depth of about one wavelength, and the particle movement is through an elliptical orbit. Lamb waves are a special case of Rayleigh waves that occur when the material is relative thin. The physical motion of surface acoustic waves of the Rayleigh-Lamb and/or Love type is associated with mechanically time-dependent elliptical displacement of the surface structure.

An “indwelling medical device” means a medical device that is left within a bodily organ or passage in a patient to maintain drainage, prevent obstruction, or provide a route for administration of food or drugs. As used herein, “indwelling medical device” includes (without limitation) implantable medical devices that act upon a cassette, reservoir, vial, syringe or tubing to convey medication or fluid to or from a patient, implantable devices for monitoring patient vitals or other parameters, or implantable diagnostic devices. In preferred embodiments, indwelling medical devices include intravenous and gastrointestinal catheters, and—specifically— intravenous (IV) catheters and percutaneous endoscopic gastrostomy (PEG) tubes.

As described in the present disclosure, provided is an innovative approach to inhibiting biofilm formation and thus preventing infection commonly associated with indwelling medical devices. High frequency acoustic waves are generated from electrically activated piezoceramic elements for homogeneously dispersing on surfaces of indwelling medical devices of varied consistencies, sizes, and shapes. The indwelling medical device effectively becomes the source of acoustic mechanical energy transmission. To achieve the effective physical energy requirements for harnessing the surface acoustic waves for preventing microbial attachment and biofilm formation, embodiments of the invention employ piezo actuators as the ultrasound transducer that generate high frequency elastic acoustic waves of non-thermal range and may be applied to a wide range of microorganisms on indwelling medical devices in vitro and in vivo.

The frequency and modes of vibrations in piezo elements (separately or in combination) may be chosen in such a away so as to achieve desirable vibration resonance, and the vibrations may be transmitted in different directions. For example, the vibrations may be transmitted in the direction of the body (towards the distal end of the device), away from the body (toward the proximal end of the device), along the longitudinal direction of the device (through its inner/outer surfaces), etc.

The frequency of transmitted waves may depend on the type of indwelling device or its constructions (e.g., material, manufacturing, etc.) and may not be the same as piezo-ceramic resonance frequency. By means of a processor, in addition to choosing the proper resonance frequency of piezo-ceramic elements, it may be possible to achieve effective vibrations on the surface of the indwelling medical device. The vibrations from the piezo element(s) and the device surfaces may also be transmitted to the liquids or materials that are in contact with the piezo elements. These liquids and materials may receive micro vibration energy, thereby preventing the formation of biofilm.

Combinations of vibration may be optimally selected from the various vibration modes discussed herein depending on the specific indwelling device, the material and manufacturing resolution of the device, and even the specific biofilm microbiology of a patient. In embodiments, a particular combination of vibration modes may be selected to be specifically optimized for a patient and for the indwelling device to be used in the patient.

High frequency, low energy “elastic waves” are generated from electrically activated piezo-ceramic elements that are designed to travel on solid or semi-solid surface and effectively prevent formation of microbial biofilms on solid surfaces of variable structures. Propagation of elastic waves can be adjusted and/or optimized to achieve even distribution on inert surfaces having different material compositions and of different shapes, specifically including (but not limited to) tube-shaped structures. In addition to longitudinal dispersion, internal, external, and cross-sectional zones of an indwelling medical device may acquire a transversal vector surrounding the surface with a corona of waves perpendicular to the surface of dispersion. The acoustic elastic wave corona is repulsive to bacteria and interferes with their docking and attachment to solid surfaces, thus disrupting the initial phases of microbial biofilm development. In view of the foregoing and as described in more detail herein, embodiments of the invention provide a system, device, and method for inhibition of bacterial biofilm formation on or around various indwelling devices to prevent infection that is frequently associated with such indwelling devices.

According to a first aspect of the invention, a portable ultrasound system comprising an energy-generating module configured to generate a driving signal that can be transformed into ultrasonic energy and comprising a power source, an oscillator, and a driver component; and an ultrasound transducer comprising a piezoelectric component, the ultrasound transducer being configured to receive the driving signal from the energy-generating module, to transform the driving signal into ultrasonic energy, and to control a direction of the ultrasonic energy emitted from the ultrasound transducer, wherein the oscillator and driver component are housed on or within the ultrasound transducer, and the power source is not housed on or within the ultrasound transducer.

According to another aspect of the invention, provided is a portable ultrasound device that be configured to include a power source, ultrasound driver, and the ultrasound transducer, wherein the portable ultrasound device can be controlled by the user as a single working unit. The portable ultrasound device may comprise at least one piezo-ceramic element and a vibration processing unit that, when configured with or coupled to the indwelling medical device, may produce vibrations or micro-vibrations that can disperse microbe colonies. In embodiments, the portable ultrasound device according to the invention may comprise a thin plate piezo transducer, which, after activation by a processor, begins to vibrate in bending vibration modes, creating standing acoustic waves on the thin plate piezo transducer. Multiple energy picks interchange with minimal energy levels on the transducer surface and act like small energy needles. Due to these energy needles, the transducer creates surface acoustic waves on the indwelling device surface, which are transmitted as running waves along the length of the indwelling device surface. The thin plate transducer may also create acoustic waves in inner channels of the indwelling device.

In embodiments, the ultrasound transducer may comprise multiple transducers, which may be optimally configured to provide SAW in multiple directions, wherein the multiple directions may further provide a particularly focused effect. A processor, such as a vibration processor, may supply electric signals, which may be transformed by the piezo-ceramic element(s) into mechanical vibrations, such as sound waves. The vibrations may cause the piezo-ceramic element to oscillate, thereby creating vibrations on the indwelling medical device surface and/or partially propagating to the relevant internal organs, cavities, passageways, etc.

The vibrations may be micro-vibrations and may be significantly amplified if a resonance condition is attained through the indwelling medical device and/or internal area. A resonance condition may cause an increase in the amplitude of oscillation of the acoustic ultrasound device when exposed to a periodic force whose frequency is equal to or very close to the natural undamped frequency of the device. This resonance may intensify and/or prolong the acoustic vibrations generated by the piezo-ceramic element(s), relative to the energy supplied by the vibration processor. The effects of resonance may further aid in the dispersal of microbe colonies that have associated around the indwelling device and/or the inner organs or of microbe colonies that are attempting to do so.

The portable ultrasound device of the invention has a size small enough to be placed inside a patch and/or coupled to or as part of an indwelling medical device. In certain embodiments, the portable ultrasound device may be a wearable ultrasound device configured to provide ultrasound energy for extended periods of time and may be used for wireless energy transfer and recharging. In still further embodiments, the ultrasound system or portable ultrasound device may be implantable within a subject as part of or operatively coupled to an indwelling medical device.

In still further aspects, the portable ultrasound system, ultrasound transducer, and portable ultrasound device of the invention may be used in methods and novel applications further described herein.

In accordance with one object of the invention, the portable ultrasound device may inhibit the formation of microbial colonies and, insofar as these microbial colonies may lead to development of harmful biofilms that lead to infections, the acoustic ultrasound device may effectively prevent biofilm-associated infections in a patient following introduction of an indwelling medical device. In this regard, provided is a method for treating a surface of an indwelling medical device to prevent colonization or attachment of bacteria thereto, the method comprising: providing an ultrasound transducer or an actuator for producing surface acoustic waves (SAW) on a surface of the indwelling medical device and a processor for controlling parameters of said transducer; activating the transducer to generate elliptical motion of particles on the surface of the medical device and thereby generate SAW, wherein the elliptical motion causes vibration of bacteria on the surface of the indwelling medical device at a particular vibration amplitude relative to vibration of the surface of the indwelling medical device; and controlling parameters of the transducer via the processor such that the vibration amplitude of the bacteria is smaller than a Z-potential repulsive zone of the surface of the indwelling medical device, such that the SAW inhibit attachment of bacteria to the surface of the medical device.

According to methods of the invention, the producing of the SAW may comprise producing waves selected from the group consisting of: Rayleigh waves, pseudo-Rayleigh waves, and Lamb waves. The producing of elliptical motion may comprise producing elliptical motion of particles on an external surface of the indwelling medical device or on an internal surface of the indwelling medical device, in a single direction or in multiple directions, simultaneously or in tandem (if in multiple directions). Furthermore, the producing of elliptical motion of surface particles causes bacteria and other particles on the surface of the indwelling medical device to move in a direction which is opposite to a direction of SAW propagation.

In embodiments, the piezo-ceramic element of the transducer may be coupled to an inner or outer surface of the indwelling device, and, as a result, vibrations from the ceramic element (thickness, longitudinal, torsion, flexural (bending)-flexural, longitudinal (radial)-flexural, radial-longitudinal, flexural (bending)-torsional, longitudinal-torsional and radial-shear modes) may be transmitted through the device material, through the inner or outer surfaces of the device, generating traveling surface acoustic waves, e.g., of Rayleigh-Lamb type and/or Love type. The frequency and amplitudes of vibrations of the piezo-ceramic element are adjusted to the shape and material of the indwelling medical device to enable creation of surface acoustic waves on inner and outer surfaces of the indwelling medical device along a longitudinal direction thereof.

The acoustic energy transmitted through the surface of the indwelling medical device may be adjusted to create mechanical micro-vibrations capable of disrupting (thus preventing) biofilm formation on surfaces of the indwelling medical device. Specifically, the energy of micro vibrations is adjusted to force the bacteria to move relative to the vibrating surface of the indwelling device. The relative motion of the bacteria in relation to the surface of the indwelling medical device results in disruption of the bacterial attachment process and influence on other components of the biofilm formation process, such as bacteria quorum sensing.

Attraction or repulsion of bacteria is an outcome of Van der Waals and hydrophobic attraction forces being counteracted by electrostatic repulsion in the 10 nm range near the surface—a phenomenon known as Z potential. Because SAW-induced elliptical vibrations and the amplitude at which the bacteria vibrate is smaller than that of the surface vibrations, energies in the bacteria-killing range are not required for inhibiting bacterial attachment to surfaces. Thus, unlike known bacterial killing methods using high energy, the method according to the invention employs preventive distribution of low acoustic energy by means of surface acoustic waves.

Although various applications of the herein described system and ultrasound device are envisioned may be obvious to those skilled in the art, indwelling medical devices suitable for application of the portable ultrasound device and methods of the invention include catheters, stents, grafts, orthopedic implants, temporary and permanent implants, stents, endoscopic devices, syringes for intravenous delivery, and PEG tubes. Irrespective of the type of indwelling medical device, typically these devices (such as, e.g., PEG tubes) are formed of materials to which bacteria adhere and, in a relatively short length of time, the bacteria adhered to the surface of said indwelling device will colonize and form a biofilm.

In embodiments, the indwelling medical device is selected from a PEG tube or an intravenous (IV) delivery system. Bacterial biofilm formation on the surface of indwelling devices is prevented due to the bacterial being forced to move relative to the SAW on the surface of the indwelling device. The relative motion of bacteria results in bacterial quorum sensing and disrupts the bacterial attachment process. The method is preventative (i.e., prophylactic) insofar as the SAW generated by the ultrasound transducer create low surface acoustic energy that disrupts said biofilm formation that leads to infection.

In certain embodiments, also provided is in improved indwelling device or, specifically, a PEG tube, and method of inhibiting biofilm formation and preventing biofilm-associated infection associated therewith. The improved indwelling medical device comprises a portable ultrasound device of the invention attached or coupled to a surface thereof. Upon activation (through a bus cable or wireless communication), the ultrasound transducer of the portable ultrasound device converts a driving signal into SAW further transmitted in one or more directions along the exterior and/or interior surface(s) of the indwelling medical device. For example, the portable ultrasound device of the invention transmits SAW of Rayleigh-Lamb and/or Love type to the exterior surface of the indwelling medical device along at least a longitudinal direction towards the distal end of the indwelling medical device. In some embodiments, surface acoustic waves generated by the ultrasound transducer propagate in at least one of two opposite directions from the transducer (piezo actuator): towards the body and towards the proximal end of the indwelling medical device. In still further embodiments, propagation of surface acoustic waves may be restricted to one direction only by means of acoustic absorbers in the form of rings, which may eliminate propagation of acoustic energy.

In certain embodiments, a SAW energy source may be coupled to an indwelling medical device near the proximal end opposite the direction of the body and a SAW-transmission transducer may be coupled to the indwelling medical device near the distal end in the direction of the body. In certain embodiments, one or more transducers may be located at the distal end that is introduced into the body or at the proximal end that remains exterior to (protruding from) the body. The SAW energy source, typically an external power source, transmits an activation signal to the transducer. Upon activation, the transducer generates low frequency, low intensity surface acoustic waves (SAW) which are then propagating throughout/along the SAW transmission member.

In but one exemplified embodiment of the invention, provided is an ultrasound device configured to generate and transmit SAW onto a surface of an indwelling medical device. An electronic driver sends periodic electrical pulses to an actuator harboring a ceramic piezo element. The frequency of the vibrations generated by the piezo element is 100 kHz+10% and at on/off frequency of 30 Hz; the acoustic intensity was 0.4 W cm⁷² and an amplitude of 2 mm. The acoustic energy on the inner surface of the catheter is 0.3 mW cm⁷² with a wave amplitude of 0.2-2 nm. In the Epsilometer test experiments, the acoustic intensity and amplitude were preserved through a 10-fold decrease in actuator energy.

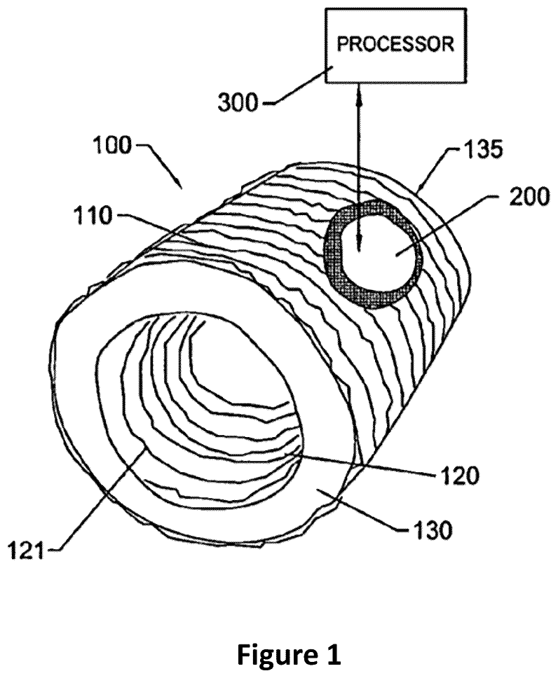

With reference being made to FIG. 1, provided is a schematic illustration of an indwelling medical device 100 with an ultrasound transducer 200 for producing surface acoustic waves according to embodiments of the invention. Indwelling medical device 100 has an external surface 110, an internal surface 120, a first or distal end 130 in the direction of the body, and a second or proximal end 135 in the direction away from the body. An ultrasound transducer 200 is attached to external surface 110 of the indwelling medical device 120 and in electrical communication with a processor 300. In certain embodiments, processor 300 may be, for example, a central processing unit (CPU), and may include an oscillator, an amplifier, and any other component conventionally used for receiving and transmitting signal and making calculations relating to the received and transmitted signals. Upon receipt of an electrical signal from processor 300, ultrasound transducer 200 is configured to generate high frequency mechanical vibrations in a range from KHz to MHz. These high frequency mechanical vibrations create surface acoustic waves (SAW) 121 (in the nanometer range) on internal surface 120 and external surface 110 of the indwelling medical device. 100. The frequency of generated mechanical oscillations in in the ultrasound transducer 200 is directly related to the frequencies produced by the processor 300. Thus, if oscillations are in the MHz range, the mechanical vibrations will also be in the MJHz range, and similarly for other ranges.

Ultrasound transducer 200 may be comprised of one or multiple piezoelectric transducers, or one more electromagnetic acoustic transducers, or one or multiple laser pulse transducers. In the case of piezoelectric and electromagnetic transducers, as used in preferred embodiments of the invention, direct contact between the ultrasound transducer 200 and indwelling medical device 100 is necessary.

Surface Acoustic Waves (SAW) generated and applied to an indwelling medical device according to the invention may be selected from various types of waves or combinations thereof. For example, a combination of Surface and Rayleigh waves generates an elliptical orbit and symmetrical mode, whereas a combination of Plate and Lamb leads to an extensional wave with a component perpendicular to the surface, a combination of Plate and Love is parallel to a plane layer and perpendicular to the wave direction, and Leaky Rayleigh Waves are guided along an interface, while Sezawa waves have an antisymmetric Mode. Surface and Rayleigh waves travel along the boundary between two different media, penetrating to a depth of about one wavelength. The particle movement has an elliptical orbit.

Attraction or repulsion of bacteria is an outcome of Van der Waals and hydrophobic attraction forces becoming counteracted by electrostatic repulsion in the 10 nm range near the surface—a phenomenon known as “Z potential.” SAW-induced elliptical vibrations, and the amplitude at which the bacteria vibrate is smaller than that of the surface vibrations. The result is a relative velocity of bacteria respective to the surface. When the SAW-generated bacterial vibration amplitudes are smaller than the Z potential repulsive zone, an overall net repulsion occurs, preventing bacterial attachment, and being effective in inhibiting particle attachment to the surfaces, inhibiting adhesion, growth and aggregation of cells into micro colonies process on the surfaces, maturation and dissemination of progeny cells for new colony formation. Increasing the bacteria vibration amplitudes to values exceeding the Z potential repulsion zone will result in a net attraction force, promoting the adhesion of bacteria. For this reason, many regimes which see to use ultrasound energy to disrupt bacterial adhesion fail.

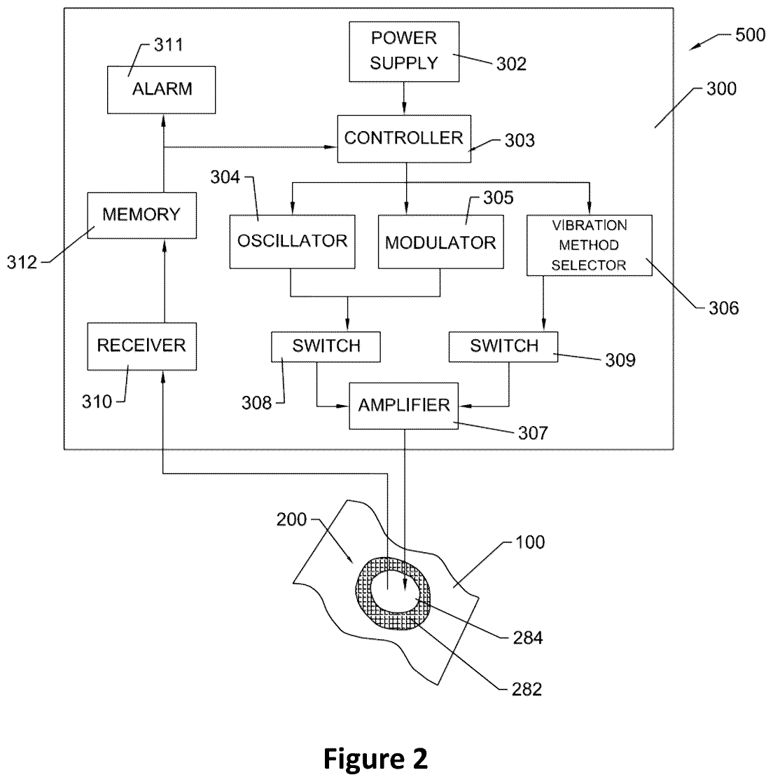

Reference is now made to FIG. 2, which shows a block diagram illustrating a system 500 for applying SAW to an indwelling medical device according to embodiments of the invention. As shown, the system 500 is useful in creating SAW via a piezoelectric transducer (actuator); however, as noted below, other methods may be used to create SAW, including electromagnetic stimulation and laser pulse excitation. System 500 includes an ultrasound transducer 200 having an activating portion 282 and an electrode portion 284, a processor 300 in electrical communication with electrode portion 284, and optionally a matching layer positioned between a transducer 200 and an indwelling medical device 100. According to certain embodiments, as shown in FIG. 2, the ultrasound transducer 200 is a piezoelectric transducer and works by converting electrical signals from processor 300 into mechanical energy, wherein the mechanical energy is transmitted to an indwelling medical device 100 and creates SAW on surfaces thereof. In some embodiments, transducer 200 is configured to transmit electrical signals proportional to the mechanical energy created by processor 300, and may thus provide a feedback loop to regulate the electrical signals produced by processor 300. The matching layer may optionally be placed between transducer 200 and indwelling medical device 100 in order to match acoustic signal transmission properties of materials used to construct the indwelling medical device 100 and transducer 200.

Processor 300 includes a power supply 302 for providing electrical energy to the system 500. In some embodiments, power supply 302 may be a separate unit (e.g., a power cord), and in some embodiments, power supply 302 may be incorporated into the processor 300 (e.g., a battery). Processor 300 further includes a controller 303 for controlling output parameters of processor 300. Controller 303 is in electrical communication with an oscillator 304 for providing signals at various frequencies, a modulator 305 for modulating parameters such as frequency, amplitude, etc., and a vibration method selector 306 for providing different types of vibrations, such as single-phase, two-phase or multi-phase vibrations. Oscillator 304 and modulator 305 are connected to a first switch 308 for selection of signal parameters. Vibration method selector 306 is connected to a second switch 309 for selection of a vibration method, and the signal of the selected vibration type is sent through an amplifier 307 to the transducer 200.

In embodiments wherein electrical signals are sent from transducer 200 to processor 300, these signals are received by a receiver 310 within processor 300. In some instances, signals may be sent by a separate sensor placed on or near or incorporated within the transducer 200, as will be described in further detail below. Signals received by receiver 310 are sent to a memory module 312 where they are compared with expected values. Results of the comparison are then either sent to controller 303 where signal parameters such as amplitude and frequency may be automatically adjusted based on the received information or sent to an alarm 311 for alerting a user that parameters should be adjusted manually.

As mentioned above, selection of parameters depends on the use and application of system 500 and may vary according to specific requirements. For example, where the indwelling medical device is a urinary catheter, three phases would be required: an insertion phase, an indwelling phase, and a removal phase. The indwelling phase may last up to 30 days and may itself include separate healing phases. Each of the phases may also have different requirements, leading to further selection of still additional parameters. During the insertion phase, for example, a two-phase electrode method may be used, wherein two electrodes working together produce larger vibration amplitudes. This type of effect is desirable, for example, during insertion of a catheter, when reduction of friction is desired. During an indwelling phase, at different points in time, different frequencies and/or amplitudes may be chosen depending on the stage of healing. For example, modulator 305 may modulate MHz signal with a KHz signal by additive synthesis, and a single-phase vibration may be chosen. Although friction reduction is not important during this phase, relative velocity of bacteria for reduction of bacterial adhesion is important. To achieve this result, smaller amplitudes could be employed, such as those obtained via a single-phase vibration scheme. Such parameters would allow for a decrease in energy requirements for inhibiting biofilm and crating relative velocity of bacteria. During the removal phase, high amplitudes are desirable so that tissue trauma can be avoided. Thus, modulator 305 may modulate a KHz signal with a Hz signal and use a multi-phase vibration method.

In further consideration of suitable parameters, the physical motion of a so-called “true SAW” wave is associated with mechanically time-dependent elliptical displacement of the surface structure (not shown). Specifically, one component of the physical displacement is parallel to the SAW propagation axis x and another component is normal to the surface along axis y. In general, the amplitude of surface displacement along the y-axis is larger than along the SAW propagation axis x. The amplitudes of both SAW displacement components are negligible for penetration depths (into the body of the solid, such as, e.g., indwelling medical device) greater than a few acoustic wavelengths.

Propagation of Lamb waves depends on density, elastic and other material properties of the indwelling medical device for example, and they are influenced by a selected frequency and material thickness. With Lamb waves, a number of modes of particle vibration are possible, but the two most common are symmetrical and antisymmetric. The complex motion of particles is similar to the elliptical orbits for surface waves.

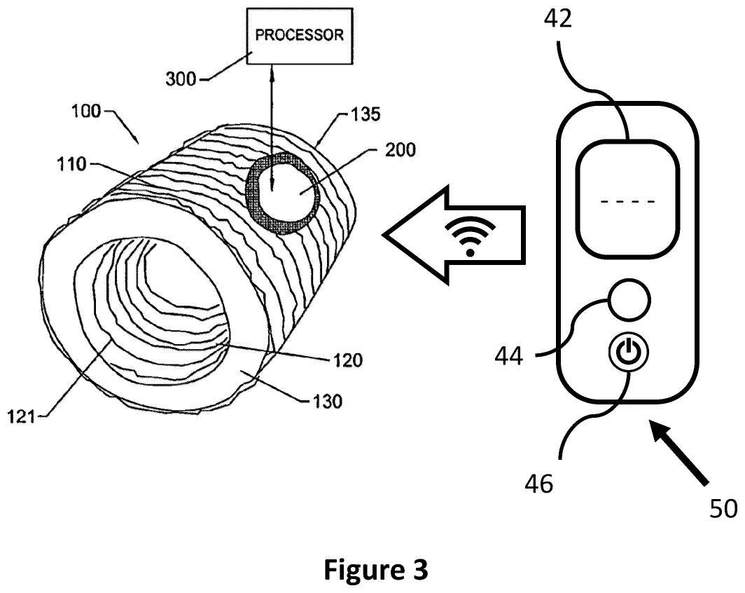

Turning to FIG. 3, illustrated is a portable ultrasound system according to embodiments of the invention. As shown, an indwelling medical device having a proximal end 130 and a distal end 135 comprises an ultrasound transducer coupled thereto, wherein the ultrasound transducer 200 receives a signal wirelessly from a power module 50. The power module 50 comprises a display window 42, a power button 46, and a control button 44 for controlling energy transmission to the ultrasound transducer 200. The power module 50 and ultrasound transducer 200 may be operably and communicatively coupled.

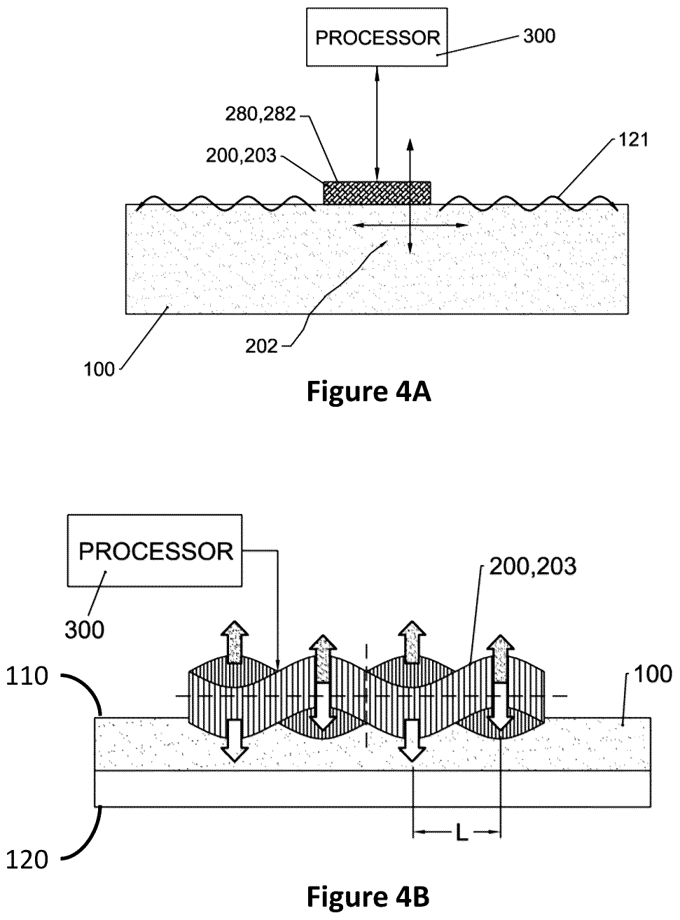

Illustrated in FIG. 4A is an ultrasound transducer 200, wherein the ultrasound transducer Is comprised of one or multiple piezo-elements 203. Transducer 200 may include a base portion 280 and an activating portion 282, wherein activating portion 282 is comprised of piezo-elements 203. It should be noted that electrodes must be included on piezo-elements 203. In many of the figures, these electrodes are not shown since the different possibilities for positioning of electrodes are known to those skilled in the art in some embodiments, base portion 280 is also the activating portion 282 and is thus comprised of piezo-elements 203. Shown in FIG. 4A is an actuator 200 comprised of a base portion 280, wherein base portion 280 is a piezo-element 203, and thus acts as an activating portion 282. In some embodiments, multiple piezo-elements 203 are used. Actuator 200 may work in thickness and/or radial vibration modes thus generating SAW 121 on surfaces of an indwelling medical device 100. Vibrations of piezo-element 203 should occur in two planes, as depicted by arrows 202.

Reference is now made to FIG. 4B, which is an illustration of an ultrasound transducer 200 such as the ultrasound transducer 200 shown in FIG. 4A, during vibrations. Transducer 200, after activation by processor 300, begins to vibrate in two directions—up and down—as shown by gray and white arrows, respectively. Vibrations of piezo-element 203 generate SAW on the internal surface 120 and external surface 110 of an indwelling medical device 100 when a distance L between two maximal amplitudes of bending vibration modes are proportional to one-half the length L of the SAW. In this embodiment, piezo-element 204 is configured to work with symmetrical Lamb vibration modes. This method works similar to the IDT 205. The standing wave maximal amplitudes created in a thin plate are similar to elongated portions 206 of IDT 205, creating elastic deformations in the material of indwelling medical device 100 and exciting SAW thereon. In some embodiments, a matching layer may be positioned between actuator 200 and indwelling medical device 100. For example, a glue layer for attaching actuator 200 to indwelling medical device 100 may be used, wherein the glue layer has a smaller acoustic velocity than piezo-element 203 but a larger acoustic velocity than the material of indwelling medical device 100.

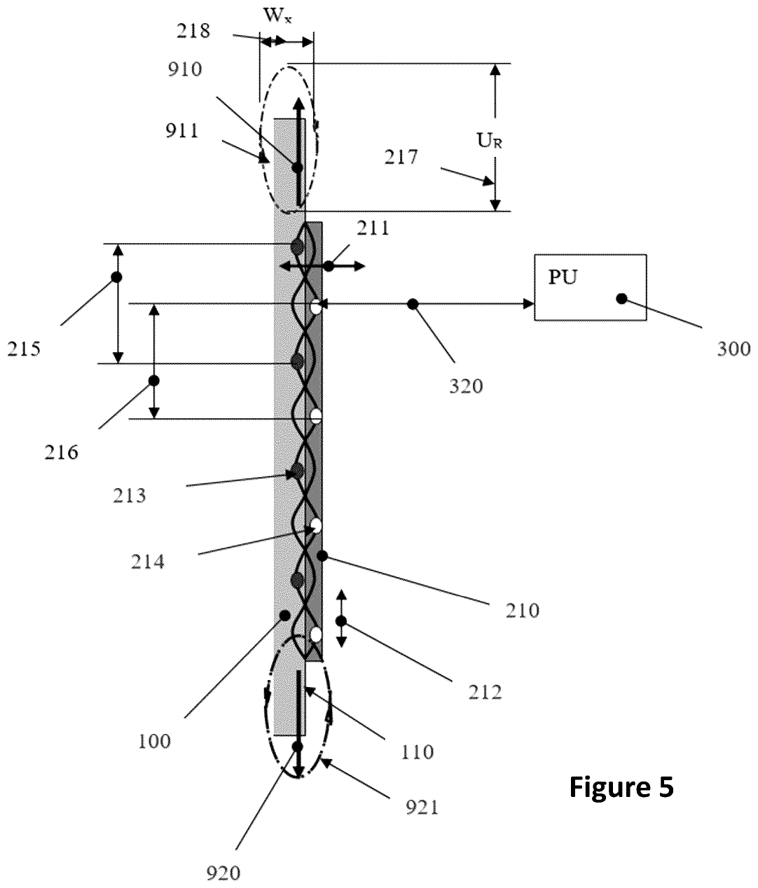

Reference is now made to FIG. 5, which is a schematic illustration of SAW generation on an indwelling medical device 100. The indwelling medical device 100 shown herein may be a PEG tube 101, but it should be readily apparent that the following description applies to various other indwelling medical devices. Ultrasound transducer 200 is comprised of a thin piezo-electric (PZT) plate element 210, which is attached to PEG tube 101. Processor 300 is a driver which provides periodic electrical pulses to PZT plate element 210, which results in mechanical vibrations in normal modes. PZT plate element 210 is attached to an external surface 110 of catheter 101 and is connected via a cable 320 to processor 300. An electrical signal from processor 300 excites bi-directional vibrations in PZT plate element 210, as shown by arrows 211 and 212. The sum of the bi-directional vibrations is a bending vibration mode, depicted by sinusoidal lines 219 having maximum points on each side, as depicted by points 213 and 214. Maximum points 213 and 214 generate mechanical vibrations on the surface of the indwelling medical device 110. A distance 215 between maximum points 213 is chosen to be equal to one-half the length of the SAW. Similarly, a distance 216 between maximum points 214 will also be equal to one-half the length of the SAW. In this way, a running wave is excited on surface 110 in directions shown by arrows 910 and 920. These SAW are low-energy and fade with depth. Their physical motion causes a time-dependent elliptical displacement of device material particles, as shown by broken arrows 911 and 921. One longitudinal vector spreads parallel to the wave propagation x axis along the surface of the catheter, triggering horizontal surface particle displacement (U_{R}) 217. The length of surface wave U_{R} is equal to two distances 215 or 216 of piezo ceramic vibration. Another transversal compression wave component (W_{R}) 218 develops on the y axis normal to the device surface causing displacement in the direction of surrounding tissues or fluid. The amplitude of this wave W_{R} is shown as distance 218. SAW excited on the device have a first direction 910 and a second direction 920 of the propagating wave and may be assumed to be Rayleigh type waves. Rayleigh type acoustic waves cause the device surface particle oscillations in directions that are parallel to the wave propagation x axis (U_{R}).

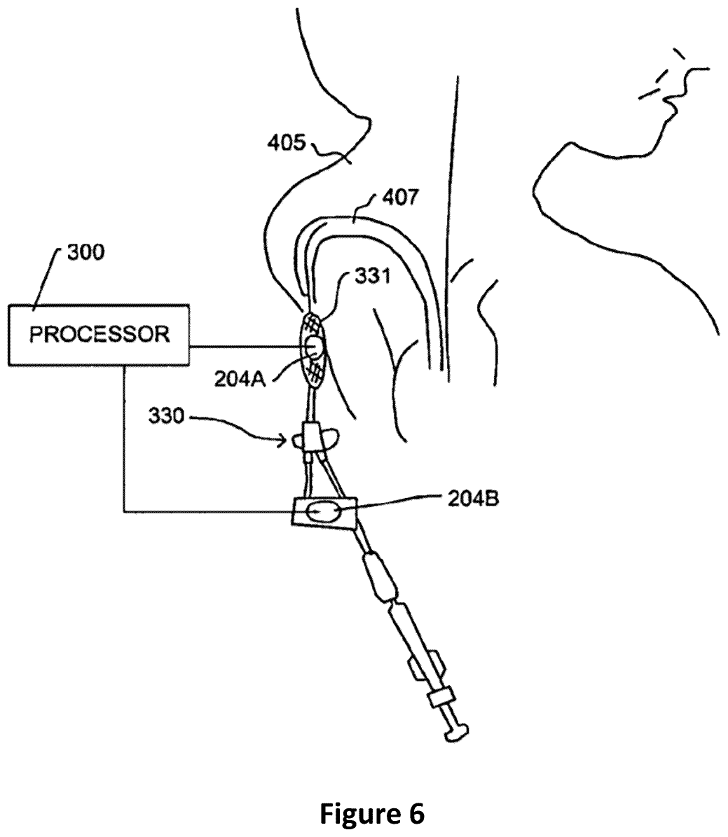

Reference is now made to FIG. 6, which is an illustration of an indwelling medical device, specifically, a central venous catheter system 330, inserted into a body 405 of a patient. Central venous catheter system 330 is threaded through a vein in the neck (the external or internal jugular vein) or a vein 407 in the upper chest under the collar bone (the subclavian vein) into a large central vein in the chest (the superior vena cava). The two general types of catheter systems which are permanently placed under the skin are internal catheters, which are completely enclosed, and external catheters, which exit through the skin By generating SAW, the vibration of the surface material of catheter system 330 results in a decrease in the coefficient of friction of the material of catheter system 330, which improves biocompatibility by reducing frictional irritation and cell adhesion at the biomaterial-tissue interface. The process of precipitation and formation of crystals (accelerated kinetically by the presence of rough surfaces, catheter holes and edges) is also reduced.

As shown in the embodiment of FIG. 6, two thin piezo-elements 204 are attached at separate locations on central venous catheter system 330. A first thin piezo element 204A is incorporated into a pad 331, and a second thin piezo element 204B is attached as a clip-on to an external portion of catheter system 330. The two piezo-elements 204 are in electrical communication with processor 300, which is located outside of the body 405. First piezo-element 204A is configured to provide an infection-free environment at the insertion site-adjacent to an internal location of catheter system 330, and to help heal the catheter insertion wound. Pad 331 may be, for example, a disinfecting pad placed at the insertion site prior to introducing the catheter. In addition, pad 331 may be left in place during healing, preventing bacterial infection during that phase. Second piezo element 204B creates SAW on internal and external surfaces of central venous catheter system 330 and possibly on the hub or connector of the system to inhibit biofilm formation in critical areas.

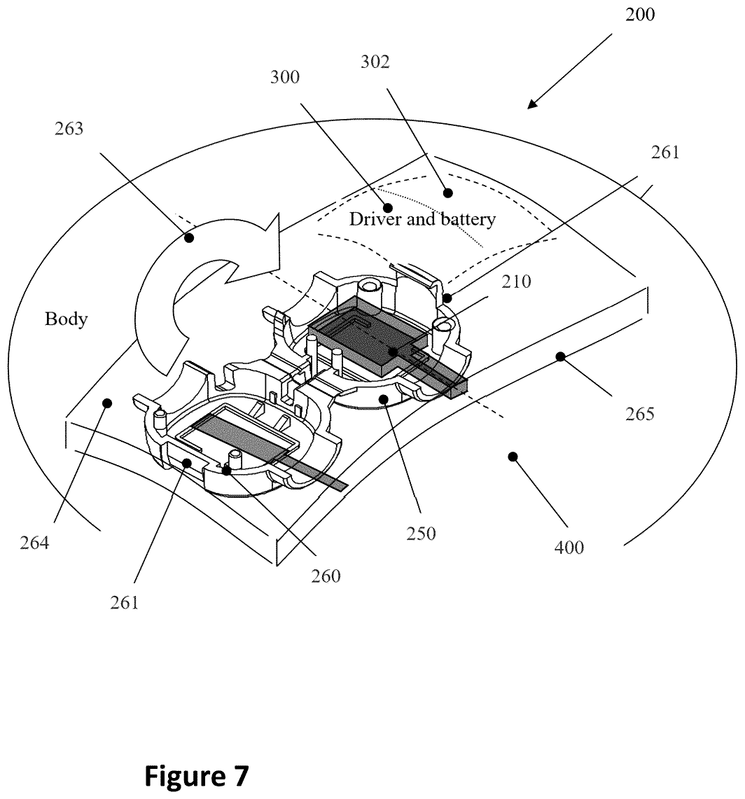

Turning to FIG. 7, illustrated is another embodiment of the invention wherein the portable ultrasound device has a flexible patch configuration wherein an ultrasound transducer (actuator) is encompassed by a detachable patch and connected with a driver, wherein vibration energy is transferred/transmitted to the surfaces of the indwelling medical device and has therapeutic impact due to acoustic energy According to the flexible patch configuration shown in FIG. 7, a flexible battery 302 is incorporated with flexible electronic unit 300 into a detachable patch having an adhesive bottom surface 265. The flexible battery 302 and flexible electronic unit 300 are configured in separate layers of the detachable patch 264. A PZT element 210 of the ultrasound transducer is contained within a plastic case 200 having two sides 260 and 250. When the flexible patch 265 is adhered to a patient's leg 400, the indwelling medical device 100 is placed into a special fitting, and a lock mechanism 261 locks the indwelling medical device to the patch.

In embodiments, a length of the portion of the indwelling medical device running between a patient's body and latch is long enough and does not result in mechanically pushing the indwelling medical device out of the body. The plastic case 200 is detachably coupled to a top or upper surface/portion of the patch 264 using an adhering mechanism such as a clasp or hook-and-loop elements or using any other known clip-on mechanisms, which allow movement of the case relative to the patch. The driver unit in a patch configuration may be a chip device or flexible CPU system, configured on the patch basis, and electrically connected with a flexible battery which is based on the same patch. This disposable device may have a rechargeable flexible battery, or all the parts including the battery may be disposable. As another advantage, the patch configuration eliminates the need of the driver and the actuator to be separate parts of the device. Driver and actuator may be integrated into one flexible part based on the securing patch. The patch according to embodiments may also enclose wireless regulation feature, enabling medical personal to switch on and off the device when it is needed, and to regulate the acoustic intensity depending on patient status, on drugs, and other considerations.

In addition to the main function, actuator may transmit acoustic energy through the patch material to the human skin under the patch. These vibrations cause micro massage at the location of adhesion, thus reducing, and perhaps eliminating, irritation of the skin and making it easier to pull the patch material off after use.

In another variation, the patch may comprise a medical grade patch, the ultrasound transducer and one of driver options (stand-alone box and chip on a side of the patient leg and on other side to the actuator using an adhering mechanism, such as Velcro® material. The actuator is electrically connected with driver box. In still another variation, the patch may comprise the transducer case incorporated therein by a special manufacturing procedure.

The patch ensures two functions: stabilizing the indwelling medical device, such as the depicted urinary catheter and preventing trauma which may occur if the catheter is pushed, and exciting SAW on the catheter surfaces, preventing biofilm formation, minimizing trauma of indwelling devices to the body tissues. The patch may be manufactured as a water-resistant device. The main requirement for this construction is to enable acoustic contact between the PZT element and the catheter surface. Such a water-resistant device may contain two parts. The first part is attachable to the body, and the catheter is secured to this part. The second part containing the transducer, driver and battery is put on the first part as a sandwich type construction. The opposite case, when the active elements are incorporated into the first part, is also possible. Other constructions are also possible, enabling the production of a disposable device. The materials for active urinary patch are those conventionally used in the patch production.

An advantage of the patch configuration is that the device is user friendly, needs no long wires connecting actuator and driver, does not disturb the movement of the patient and secures the device to the patient body, preventing the possibility of the actuator slipping on the catheter surface. In addition, the patch configuration solves the problem of possible abrasion to the patient's skin, which may be caused when the catheter with the add-on device is mechanically pulled due to patient or care personal activity.

According to exemplified embodiments of the invention described herein, surface acoustic waves cause micro-motion of particles (i.e., bacteria) and liquids on an external surface of an indwelling medical device. SAW may be generated by an ultrasound transducer and applied to the surface of the indwelling medical device in a first direction, and the physical motion of SAW is associated mechanically a time-dependent elliptical displacement of surface material particles. In embodiments, SAW may cause micro-motion of particles and liquids on external surface of the indwelling medical device in a second direction that is along the same axis but in an oppositive direction to the first. This phenomenon, created by SAW, is characteristic to all materials and is effective until a depth or distance is reached. The ultrasound transducer effectively creates an acoustic energy transmission online towards the liquid and body tissues of the subject in acoustic contact with device 10. The energy transmission may have two components: (a) at a depth equal to two surface wave lengths towards the body tissue 400, the tissue particles being mechanically-elliptically oscillating, with velocities of tenths meter/second; (b) at a depth exceeding two surface wave lengths towards body tissue, the particles oscillating linearly-mechanically with nanometer amplitudes.

What is claimed is:

1. A portable ultrasound system comprising:
an energy generating module operative to generate a driving signal that can be transformed into ultrasonic energy, wherein said energy generating module comprises a power source, an oscillator, and a driver component; and
an ultrasound transducer comprising a piezoelectric component, said ultrasound transducer being operative to receive the driving signal from the energy generating module, to transform the driving signal into ultrasonic energy, and to control a direction of the ultrasonic energy emitted from the ultrasound transducer,
wherein the oscillator and driver component are housed on or within the ultrasound transducer, and the power source is not housed on or within the ultrasound transducer.

2. The portable ultrasound system according to claim 1, wherein the ultrasound energy from the transducer is emitted as pulsed, continuous, or both pulsed and continuous ultrasonic energy.

3. The portable ultrasound system according to claim 1, wherein the energy generating module comprises a voltage controller operative to control power distribution from the power source to the oscillator and driver component.

4. The portable ultrasound system according to claim 3, wherein the voltage controller comprises an on/off controller coupled to a transistor switch.

5. The portable ultrasound system according to claim 1, wherein the energy generating module and the ultrasound transducer are operatively connected and the driving signal is wirelessly communicated to the ultrasound transducer without a bus.

6. An assembly of an indwelling medical device and the portable ultrasound system according to claim 1, wherein the indwelling medical device is selected from a PEG tube an IV catheter and comprises the portable ultrasound transducer coupled to an exterior surface thereof.

7. The assembly according to claim 6, wherein the wherein the ultrasound transducer comprises a plurality of ultrasound transducers configured to supply SAW in a plurality of directions.

8. A portable ultrasound device comprising:
a power source;
an ultrasound driver; and
an ultrasound transducer configured to receive and convert a driving signal from the ultrasound driver into low frequency surface acoustic waves,
wherein the portable ultrasound device is comprised by an indwelling medical device and the surface acoustic waves generated by the ultrasound transducer are transmitted along a surface of the indwelling medical device.

9. The portable ultrasound device according to claim 8, wherein the portable ultrasound device is coupled to an exterior surface of the indwelling medical device and transmits the surface acoustic waves along an exterior surface of the indwelling medical device in an elongated direction thereof.

10. The portable ultrasound device according to claim 8, wherein the indwelling medical device is an intravenous (IV) catheter or a PEG tube.

11. The portable ultrasound device according to claim 8, wherein the ultrasound transducer comprises a plurality of ultrasound transducers.

12. The portable ultrasound device according to claim 8, comprising a thin plate piezo transducer in direct contact with a surface of the indwelling medical device, wherein the thin plate piezo transducer is configured to vibrate in bending vibration modes to create standing acoustic waves upon activation by a processor.

13. The portable ultrasound device according to claim 8 configured within a patch for adhesion of an indwelling medical device to a patient.

14. The portable ultrasound device according to claim 8, wherein the ultrasound transducer is implantable within a subject as part of an indwelling medical device.

15. An indwelling medical device for implantation in a subject's body, the indwelling medical device having an elongated tube shape comprising a proximal end opposite a direction of the body and a distal end in the direction of the body, and comprising the portable ultrasound system of claim 8 operatively coupled thereto, wherein the portable ultrasound transducer is configured within a surface of the indwelling medical device.

16. An indwelling medical device comprising the portable ultrasound device according to claim 8 operatively coupled thereto, wherein the ultrasound transducer comprises a piezoelectric element in direct contact with the surface of the indwelling medical device and, upon receipt of the electrical signal, generates high frequency mechanical vibrations to create surface acoustic waves in the nanoscale range on an internal surface and external surface of the indwelling medical device.

17. A method for inhibiting formation of bacterial biofilm associated with an implanted medical device, comprising:
providing an indwelling medical device, an ultrasound transducer positioned on the indwelling medical device, wherein the ultrasound transducer comprises a piezoelectric material in direct contact with a surface of the indwelling medical device, and a processor in electrical communication with the ultrasound transducer;
positioning at least a distal end of the indwelling medical device in a body of a subject;
activating the ultrasound transducer by powering the processor, wherein activating the ultrasound transducer produces elliptical motion of particles and generates surface acoustic waves along a surface of the indwelling medical device; and
controlling parameters of the ultrasound transducer via the processor such that a vibration amplitude of the bacteria is smaller than a X-potential repulsive zone of the bacteria.
